(19) United States
(12) Patent Application Publication
(10) Pub. No.: US 2026/0285684 A1
(43) Pub. Date: Sep. 24, 2026

(54) ION SUBSTITUTED CALCIUM PHOSPHATE PARTICLES

(71) Applicant: Psilox AB, Uppsala (SE)

(72) Inventors: Wei XIA, Uppsala (SE); Camilla BERG, Uppsala (SE); Erik UNOSSON, Uppsala (SE)

(73) Assignee: Psilox AB, Uppsala (SE)

(21) Appl. No.: 19/693,675

(22) Filed: Jun. 1, 2026

Related U.S. Application Data
(62) Division of application No. 18/127,696, filed on Mar. 29, 2023.
(60) Provisional application No. 63/329,981, filed on Apr. 12, 2022.

(51) Int. Cl. C01B 25/32; A61K 6/20; A61K 6/30; A61K 6/838

(52) U.S. Cl. CPC C01B 25/32; A61K 6/20; A61K 6/30; A61K 6/838; C01P 2002/02; C01P 2004/34; C01P 2004/51

(57) ABSTRACT

The present invention relates to hollow calcium phosphate particles comprising a respective shell comprising calcium, phosphate, water, magnesium, and strontium. The particles have a mean diameter ranging from 400 nm to 1.5 μm. The invention also relates to methods of producing such particles and compositions comprising such particles.

### RELATED APPLICATION DATA

This application is a divisional application of U.S. application Ser. No. 18/127,696, filed Mar. 29, 2023, which claims the benefit of priority to U.S. provisional application No. 63/329,981, filed Apr. 12, 2022, the entire contents of each of these applications is incorporated herein by reference.

### FIELD OF THE INVENTION

The present invention relates to ion substituted calcium phosphate particles, to methods of producing such particles and uses thereof.

### BACKGROUND

Dentin hypersensitivity is a clinical condition that can cause significant oral discomfort and pain, triggered by e.g., thermal, mechanical or evaporative stimuli. The underlying cause for the condition is that the dentin tubules have become exposed due to gingival recession or loss of enamel, in turn caused by e.g., demineralization, abrasion or erosion, excessive tooth brushing or flossing, pocket reduction surgery or as a secondary reaction to periodontal disease.

Demineralization of dentin and enamel may be countered by remineralization, in which calcium and phosphate ions present in saliva can deposit to form new mineral. This requires a local supersaturation of ions and preferably a pH above neutral to form hydroxyapatite. In order to regain mineral and tip the scale towards remineralization, additional calcium and phosphate ions are required.

WO 2021/086252 discloses spherical and hollow calcium magnesium phosphate particles and compositions comprising such particles.

U.S. Pat. No. 9,205,035 B2 discloses a method for formation of spherical particles of ion substituted calcium phosphate. The method is based on precipitation of particles from a buffered solution under static, stirring, or hydrothermal conditions.

There is still a need for improved calcium phosphate particles that could be used to prevent or treat the above-mentioned dental disorders.

### SUMMARY

The present invention aims to solve the problems of the prior art by providing hollow calcium phosphate particles.

A first aspect of the invention relates to hollow calcium phosphate particles having a mean diameter ranging from 400 nm to 1.5 μm. The hollow calcium phosphate particles comprise a respective shell comprising calcium, phosphate, water, magnesium, and strontium. The Ca:P atomic ratio is 0.9-1.3, the Sr²⁺ content is 1-6 wt %, the Mg²⁺ content is 5-10 wt % and the Ca²⁺ content is 15-25 wt %.

In one embodiment of the invention, the Ca:P atomic ratio in the respective shells is 0.9-1.3, the Sr²⁺ content in the respective shells is 1-6 wt %, the Mg²⁺ content in the respective shells is 5-10 wt %, and the Ca²⁺ content in the respective shells is 15-25 wt %.

In one embodiment of the invention, the particles are X-ray amorphous. In one embodiment of the invention, the mean diameter of the particles ranges from 700 nm to 1.5 μm.

In one embodiment of the invention, the shell has a mean thickness ranging from 20 to 300 nm, preferably around 250 nm.

In one embodiment of the invention, the particles are substantially spherical.

In one embodiment of the invention, the water is bound water.

An aspect of the invention relates to a composition comprising a paste-forming compound and hollow calcium phosphate particles.

In one embodiment of the invention, the paste-forming compound is selected from the group consisting of glycerol, triglyceride, polyethylene glycol, propylene glycol, polypropylene glycol, polyvinyl alcohol, mineral oil, liquid paraffin, and any mixture thereof, preferably glycerol.

A third aspect of the invention relates to a method of manufacturing hollow calcium phosphate particles. The method comprises forming a first salt solution comprising 0.5-1.5 mM calcium, 0.2-1.0 mM strontium, and 0.2-1.0 mM magnesium, and a second salt solution comprising 5.0-15 mM phosphate. The method also comprises mixing the first and the second salt solutions to form a mixed solution at 15-25° C., and heating the mixed solution to equal to or above 70° C. at a rate of 1-20° C./min forming a heated solution. The method further comprising precipitating particles from the heated solution at 70-100° C. for a duration of 1 min to 24 h.

In one embodiment of the invention, the method additionally comprises retrieving and washing the particles.

In one embodiment of the invention, heating the mixed solution comprises heating the mixed solution to a temperature selected within a range of from 70 to 100° C.

In one embodiment of the invention, heating the mixed solution comprises heating the mixed solution to a temperature selected within a range of from 70 to 100° C. for a duration of 5 min to 3 h.

In one embodiment of the invention, the first salt solution comprises 0.9 mM calcium, 0.6 mM strontium and 0.5 mM magnesium.

In one embodiment of the invention, the second salt solution comprises 10 mM phosphate.

In one embodiment of the invention, the ratio between Ca:P in the mixed solution is around 0.09.

A fourth aspect of the invention relates to a method of treating or preventing caries in a subject. The method comprises administering hollow calcium phosphate particles or a composition to a surface of at least one tooth of the subject.

A fifth aspect of the invention relates to a method of treating or preventing dentin hypersensitivity in a subject. The method comprises administering hollow calcium phosphate particles or a composition to a surface of at least one tooth of the subject.

A sixth aspect of the invention relates to a method of treating or preventing enamel demineralization or decalcification defects, such as white spot lesions, in a subject. The method comprises administering hollow calcium phosphate particles or a composition to a surface of at least one tooth of the subject.

A seventh aspect of the invention relates to a dental product comprising hollow calcium phosphate particles or a composition. The dental product selected from the group consisting of a toothpaste, a dentifrice, a dental varnish, a desensitizing gel, a mineralizing gel, a tooth whitening gel, a tooth whitening strip, a dental prophy paste, a mouthwash, a tablet, a chewing gum, a dental sealant, a dental filling material, a dental cement, and a dental pulp capping material.

In the following, the invention will be described in more detail, by way of example only, with regard to non-limiting embodiments thereof, reference being made to the accompanying drawings.

### LIST OF FIGURES

FIGS. 1A-1F are scanning electron microscope (SEM) images of embodiments according to the invention.

FIGS. 2A-2B are SEM images of embodiments according to the invention.

FIG. 3 is a flow-chart according to an embodiment of the invention.

FIGS. 4A-4C are SEM images of embodiments according to the invention.

FIGS. 5A-5C are SEM images of embodiments according to the invention.

### DEFINITIONS AND ABBREVIATIONS

‘Dentin hypersensitivity’—refers to dental pain arising from exposed dentin surfaces on teeth in response to a stimuli, e.g., thermal stimuli. Dentin hypersensitivity is also referred to as ‘sensitive teeth’;

‘bound water’—refers to water of hydration associated with the amorphous calcium phosphate particles according to the invention, based on the generalized chemical formula of amorphous calcium phosphate as CaₓH_{y}(PO₄)_{z}·nH₂O, n=3-4.5;

‘X-ray amorphous’—refers to a material or particles that lacks or lack long range crystalline order. The crystallinity or X-ray or XRD amorphous state of the particles is determined by powder X-ray diffraction (XRD). A crystalline material reflects the X-rays according to the arrangement of its crystallographic planes and generates an identifiable pattern of sharp peaks, whereas an X-ray amorphous material only generates a single broad diffuse peak. Herein, the particles are, thus, classified as X-ray amorphous if the generated pattern lacks identifiable sharp peaks and is only characterized by a broad diffuse peak;

‘ACP’—is short for amorphous calcium phosphate;

‘wt %’—refers to weight percent of the ingredient in relation to the total weight of the particles or the composition; and

‘HA’—refers to hydroxyapatite, chemical formula Ca₅(PO₄)₃(OH) but usually written as Ca₁₀(PO₄)₆(OH)₂.

### DETAILED DESCRIPTION

Different calcium phosphate technologies and particles have been introduced in oral care products to promote remineralization of enamel as well as occlusion of dentin tubules for reduction of dentin hypersensitivity. One of the most effective approaches is to use amorphous calcium phosphate (ACP), which, with its high aqueous solubility, can supply a high concentration of calcium and phosphate ions, making it highly bioactive. The metastable nature of ACP also means that it is easily transformed into more stable calcium phosphate phases, such as HA. In fact, ACP is considered a precursor of natural apatite in teeth and therefore plays an important role in endogenous mineralization.

A challenge in utilizing ACP in biomedical applications, such as dentin and enamel mineralization and tubule occlusion, is to stabilize it in production and product formulations. In this regard, stability refers to stability of the amorphous state in order to prevent the ACP particles from crystallizing. Aqueous formulations with ACP and long-term storage of ACP formulations tend to crystallize the material, making it less bioactive. A way of stabilizing ACP is to substitute part of the calcium with magnesium, which, with its smaller ionic radius, will disrupt the active crystallite growth sites. This has been shown to reduce the crystallization rate of ACP. The formed particles may be referred to as ion substituted calcium phosphate particles.

Strontium is chemically closely related to both calcium and magnesium. The effect of strontium in bone formation and dental applications has been widely studied.

The present invention provides ion substituted calcium phosphate particles comprising magnesium and strontium, as well as a method for producing the particles. The particles are stable upon storage, in the sense that they at least partly maintain their morphology. The as-synthesized particles are stable during storage for at least a time period of a month. The particles may be applied in oral care products, such as toothpastes, dentifrices, fluoride varnish or desensitizing/mineralizing gels to prevent or treat caries, dentin hypersensitivity and/or white spot lesions.

A first aspect of the invention relates to hollow calcium phosphate particles having a mean diameter ranging from 400 nm to 1.5 μm. The particles comprise a respective shell comprising calcium, phosphate, water, magnesium, and strontium. The Ca²⁺, Mg²⁺ and Sr²⁺ concentrations in the shells are 15-25 wt %, 5-10 wt % and 1-6 wt %, respectively, and the Ca:P atomic ratio is 0.9-1.3.

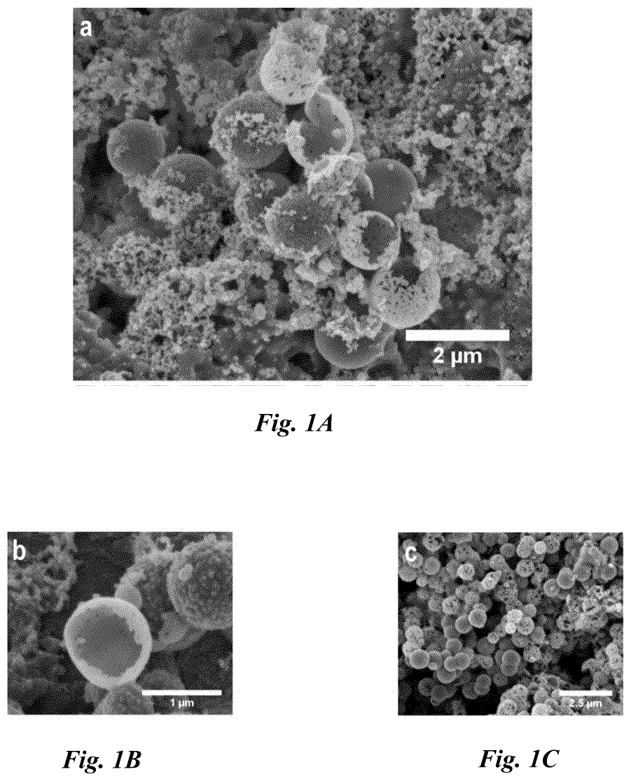

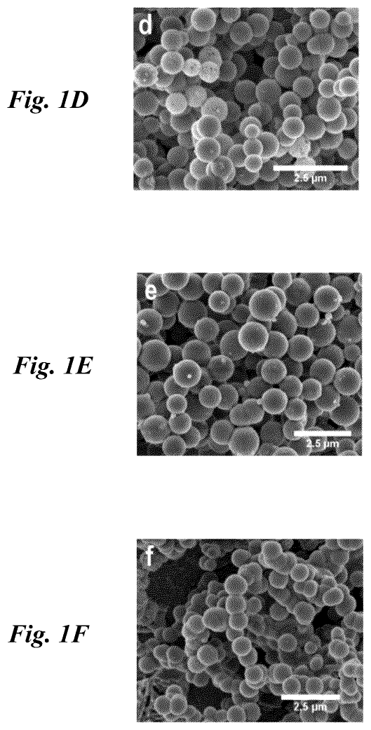

FIGS. 1A-1F show SEM images of particles according to the invention. FIGS. 1A-1F shows particles formed after different synthesis times, (1A-1B) 5 min, (1C) 40 min, (1D) 75 min, (1E) 2 hours, and (1F) 24 hours. As can be seen in the images a possible formation mechanism for the particles is that they form via self-assembly of primary nanoparticles (NPs). As can further be seen in the figures the particles typically have a diameter of 700 nm-1.5 μm. The primary NPs have a size of 20-40 nm.

FIGS. 1A-1F shows the effect of synthesis time on the particles, as can be seen the number of complete, or closed, core-shell particles increase with time. The surface structure varied from rougher after shorter synthesis, FIGS. 1A-1B, to smoother after longer synthesis time, FIGS. 1C-1F.

As mentioned above, the hollow calcium phosphate particles have a mean diameter ranging from 400 nm to 1.5 μm. This means that the mean or average diameter of individual hollow calcium phosphate particles is within the range of from 400 nm up to 1.5 μm. Accordingly, a collection of a plurality of such hollow calcium phosphate particles will have mean diameters ranging from 400 nm to 1.5 μm.

In one embodiment, the hollow calcium phosphate particles have a mean diameter ranging from 700 nm to 1.5 μm.

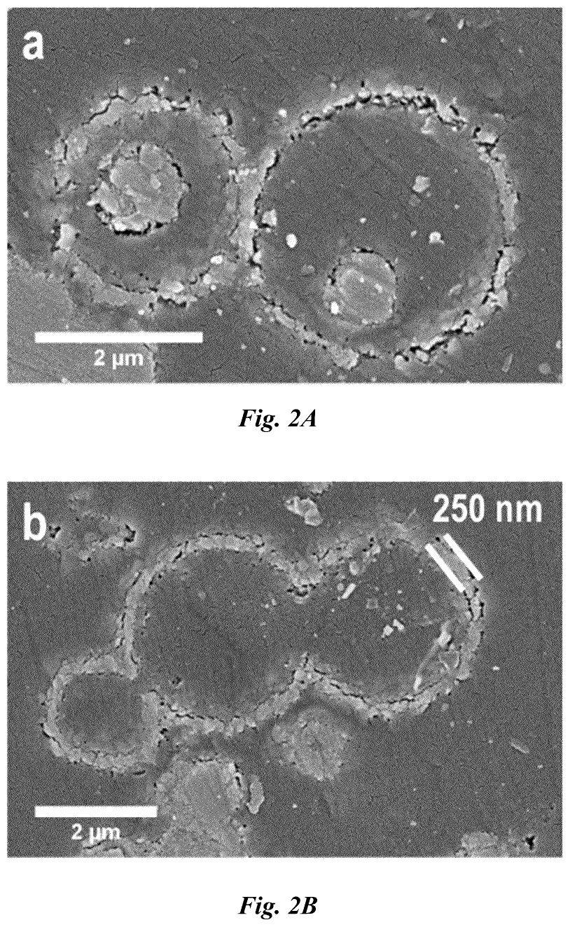

FIGS. 2A-2B show SEM images of cross-sections of particles according to the invention. As can be seen, the particles are composed of a hollow core surrounded by a shell. In one embodiment the shell thickness of the particles is 20-300 nm, preferably 200-300 nm, more preferably 250 nm, as can be seen in FIG. 2B. This suggests that the shells comprise several layers of primary NPs.

In one embodiment, the particles are substantially spherical. It is an advantage with the invention that the particles are well suited in size and shape to penetrate exposed dentin tubules.

In one embodiment, the particles are X-ray amorphous. Without being bound by theory, the amorphous character of the particles is believed to be caused by ion substitution and the conditions during particle precipitation. Ion substitution refers to the incorporation of Mg²⁺ and Sr²⁺ in the structure where Ca²⁺ otherwise would have been arranged. The ion substitution is believed to stabilize the amorphous phase of the calcium phosphate particles and thereby prolong the lifetime of the particles.

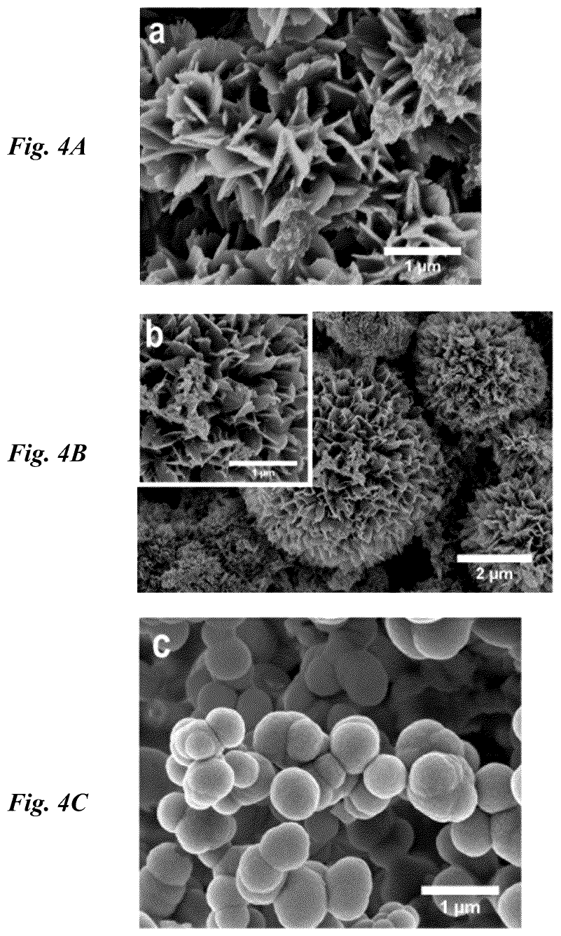

Without being bound by any theory it is possible that the ionic radii of the substituting ion influence the stability of the calcium phosphate particles. It might therefore not be possible to use any type of ion as a substitute in order to stabilize calcium phosphate particles. FIGS. 4A-4C show an example of the influence of ionic radii of the substituting ion on the calcium phosphate particles. FIG. 4A shows a SEM micrograph of crystallized particles synthesized without any substituting ions showing a morphology similar to HA. FIG. 4B shows a SEM micrograph of crystallized particles synthesized with only Sr²⁺ as the substituting ion, also showing a morphology similar to HA. FIG. 4C shows an SEM micrograph of calcium phosphate particles synthesized with only Mg²⁺ as the substituting ion, showing formation of core-shell particles. The present invention demonstrates that it is possible to synthesize core-shell calcium phosphate particles comprising both Sr²⁺ and Mg²⁺, see FIGS. 1A-1F.

In one embodiment, the water in the composition is bound water, i.e., water that is part of the chemical composition. Bound water can also be referred to as water that is incorporated within the structure of calcium phosphate particles.

A second aspect of the invention relates a composition comprising a paste-forming compound and hollow calcium phosphate particles according to the invention.

To obtain a stable composition, particles according to the invention are mixed with a paste-forming compound, for instance glycerol, then dried to remove any excess water. In one embodiment, the paste-forming compound is essentially water-free, such as <10 wt % water, preferably <5 wt % water and more preferably <1 wt % water.

It is an advantage with a composition according to the invention that the particles can be homogenously dispersed in the composition. It is further advantageous that the composition has good flowability and/or viscosity and can easily be mixed with other ingredients to prepare, for example, a toothpaste. Furthermore, by introducing the particles into a composition without a preceding drying step a narrow size distribution can be maintained by not letting the particles agglomerate into larger clusters.

In one embodiment, the paste-forming compound is selected from the group consisting of glycerol, triglyceride, polyethylene glycol, propylene glycol, polypropylene glycol, polyvinyl alcohol, mineral oil, liquid paraffin, and any mixture thereof.

Particles according to the invention can also be delivered as a slurry, i.e., in the form of a mixture of a solvent, or mixture of solvents, and particles. The solvent can, for example, be ethyl alcohol, isopropyl alcohol, or a mixture of those.

A third aspect of the invention relates to a method of manufacturing calcium phosphate particles that are ion substituted with Sr²⁺ and Mg²⁺. In short, two salt solutions, one containing the constituent cations (Ca²⁺, Mg²⁺, and Sr²⁺) and one containing the anions (H₂PO₄⁻/HPO₄²⁻) are mixed forming a mixed solution at or slightly less than room temperature (20-25° C.). The mixed solution is heated to above 70° C., after which precipitates start to form in the heated solution. The precipitates are collected after 1 min or up to 24 hours by for example vacuum filtration, and thereafter optionally washed with e.g., deionized water and/or ethanol.

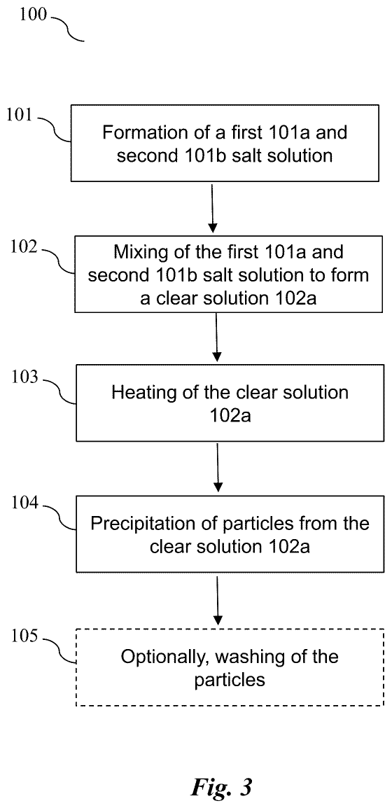

In other words, a method for manufacturing particles 100 according to the invention comprises, see FIG. 3, forming, at step 101, a first salt solution 101a comprising 0.5-1.5 mM calcium, 0.2-1.0 mM strontium, and 0.2-1.0 mM magnesium, and a second salt solution 101b comprising 5.0-15 mM phosphate. The method 100 also comprises mixing, at step 102, the first 101a and the second 101b salt solutions to form a mixed solution 102a at 15-25° C. The method 100 further comprises heating, at step 103, the mixed solution 102a to equal to or above 70° C. at a rate of 1-20° C./min forming a heated solution 103a. The method 100 additionally comprises precipitating, at step 104, particles from the heated solution 103a at 70-100° C. for a duration of 1 min to 24 h.

In one embodiment, the method 100 also comprises retrieving and washing, at step 105, the particles.

In one embodiment, the mixed solution 102a is clear, hence no or only a minor amount of precipitates are formed in the solution until it is heated to 70° C.

In one embodiment, the temperature in step 103 during heating of the mixed solution is 70-100° C.

In one embodiment, the mixed solution 102a is heated in step 103 to 70-100° C. for a duration of 5 min to 3 h.

In one embodiment, the first salt solution 101a comprises 0.9 mM calcium, 0.6 mM strontium and 0.5 mM magnesium. In one embodiment the second salt solution 101b comprises 10 mM phosphate.

In one embodiment, the ratio between Ca:P in the mixed solution 102a is around 0.09.

Without being bound by any theory, the formation of core-shell calcium phosphate particles comprising Sr and Mg can be the result of a simultaneous formation of primary NPs of calcium phosphate particles and gas bubbles of O₂, and/or N₂, and/or CO₂. The gas bubbles may be ultrafine. It can be explained by the instability of gas bubbles driving adsorption of the NPs onto the bubble surface and the gas bubbles may therefore function as soft templates in the synthesis.

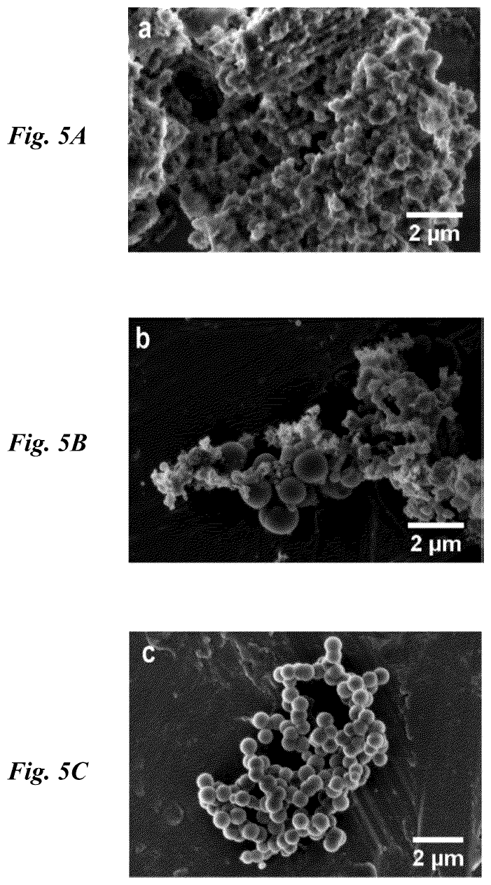

Furthermore, without being bound by any theory, the solubility of the gas bubbles discussed above are temperature dependent. It is therefore possible that at too low synthesis temperatures there is a decreased or no formation of core-shell shaped calcium phosphate particles. FIGS. 5A-5C show SEM images of particles synthesized at different temperatures. As can be seen the morphology of the particles differ between the different images, FIGS. 5B-5C show core-shell particles while FIG. 5A does not show any core-shell particles. FIG. 5A shows particles synthesized at 60° C., FIG. 5B shows particles synthesized at 70° C., and FIG. 5C shows particles synthesized at 80° C.

In one embodiment, magnesium and strontium substituted calcium phosphate particles are formed at a temperature above 70° C.

A further aspect of the invention relates to the particles or a composition according to the present invention for use as a medicament. The present invention also relates to the particles or a composition according to the invention for use in prevention or treatment of caries, dentin hypersensitivity or enamel demineralization/decalcification defects, such as white spot lesions.

‘Treatment’ of caries, dentin hypersensitivity or white spot lesions as used herein does not necessarily mean curative treatment of caries, dentin hypersensitivity or white spot lesions but also encompass inhibition or reduction of the short- and long-term symptoms of the caries, dentin hypersensitivity or white spot lesions. Hence, ‘treatment’ also encompasses delaying onset of the caries, dentin hypersensitivity or white spot lesions, including delaying, preventing onset of symptoms or resolving established pathologies associated with caries, dentin hypersensitivity or white spot lesions, or any other demineralization or decalcification defect.

The present invention also relates to a method of prevention or treatment of caries, dentin hypersensitivity and/or white spot lesions. The method comprises administering hollow calcium phosphate particles or a composition of the invention to a subject to a surface of at least one tooth of the subject.

The subject is a mammalian subject, and preferably a human subject. The particles or composition is administered locally in the oral cavity of to the subject, and in particular to the teeth of the subject, in the form of a toothpaste, dentifrice, whitening gel, dental varnish or similar product. Particles according to the present invention can be added as an ingredient in a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material.

An aspect of the invention relates to a dental product selected from the group consisting of a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material. The dental product comprises hollow calcium phosphate particles or a composition of the invention.

All embodiments disclosed herein relate to all aspects of the present invention and all embodiments may be combined unless stated otherwise.

### Examples

### Materials

CaCl₂·2H₂O, MgCl₂·6H₂O, Sr(NO₃)₂, Na₂HPO₄, and KH₂PO₄ were used as received from the manufacturer without any purification.

### Synthesis of Core-Shell Particles

Synthesis of the core-shell particles of calcium phosphate was performed based on previously published methods for synthesis using precipitation reactions in aqueous solutions (Berg et al. ‘Ion substitution induced formation of spherical ceramic particles’ Ceramics International (2019) 45:10385-10393, and Xia et al. ‘Synthesis and release of trace elements from hollow and porous hydroxyapatite spheres’ Nanotechnology (2011) 22:1-10). In short, two salt solutions, one containing the constituent cations (Ca²⁺, Mg²⁺ and Sr²⁺) were mixed with a solution containing the anions (H₂PO₄⁻/HPO₄²⁻), forming a clear solution. The reaction solutions were heated to 60-100° C. Precipitates were collected by vacuum filtration after 5 min-24 hours of heating, followed by washing once with deionized (DI) water and three times with ethanol to remove any salt residues. The reaction conditions are listed in Table 1.

TABLE 1
Experimental conditions used in the synthesis of
hollow-core shell particles in aqueous solutions.

| | Salt concentrations (mM) | | | | Ca/P | | Temperature | |
|---|---|---|---|---|---|---|---|---|
| Sample | PO₄³⁻ | Ca²⁺ | Sr²⁺ | Mg²⁺ | ratio | Time | (° C.) | pH |
| Temperature | 10 | 0.9 | 0 | 0.5 | 0.09 | 24 h | 60, 70, | 7.4 |
| | | | | | | | 80, 100 | |
| Time | 10 | 0.9 | 0.6 | 0.5 | 0.09 | 5 min, | 100 | 7.4 |
| | | | | | | 40 min, | | |
| | | | | | | 75 min, | | |
| | | | | | | 2 h, 24 h | | |
| Influence of | 10 | 0.9 | 0, 0.6 | 0, 0.5 | 0.09 | 24 h | 100 | 7.4 |
| Sr²⁺ vs. Mg²⁺ | | | | | | | | |

### Characterization

Scanning electron microscopy (SEM; Zeiss Leo 1550/1530) was used for evaluation of the morphology of the primary NPs and core-shell particles. An acceleration voltage of 5 kV and secondary electrons was used for imaging. Samples were sputtered with a conductive Au/Pt layer to allow for imaging and to avoid charging the samples.

The crystal structure and the phase evolution of the samples were analyzed with X-ray Diffraction (XRD; Bruker, D8 Advanced) using Cu Kα-radiation (λ=1.5418 Å). Samples were prepared by dispersing dried precipitates in ethanol, dropping the dispersion onto zero-background silicon sample holders where it was left to dry prior to analysis.

### Results

Different reaction temperatures between 60-100° C. were evaluated to investigate how it influenced the precipitation of calcium phosphate and the formation of core-shell particles. As shown in FIGS. 5A-5C, it was only temperatures equal to or above 70° C. (FIGS. 5B-5C) that resulted in the formation of core-shell particles. The pH remained more or less constant throughout the reactions, which could be explained by the buffering capacity of the combination phosphates (Na₂HPO₄ and KH₂PO₄) that were used in the study. The constant pH may have been an important factor for the formation of core-shell particles, but it excluded the possibility of using it as an indicator for different reaction steps (such as precipitation of ACP and formation and crystallization of e.g., HA) in the synthesis.

The formation and morphological evolution of core-shell particles synthesized at 100° C., using both Sr²⁺ and Mg²⁺, was followed by collecting precipitates at different time points during the reaction as indicated in Table 1. In the early stages of the reaction, after 5 min, it was clear that the core-shell particles, having diameters of 700 nm-1.5 μm, were constructed of primary NPs with diameters of ~20-40 nm, see FIGS. 1A-1B. After 40 min (FIG. 1C) and 75 min (FIG. 1D), there were still signs of the primary NPs assembling around hollow cores, but an increasing number of particles had complete shells enclosing the core. After the comparison between the samples from 5 and 40 min, it was apparent that the number of complete core-shell particles increased with time. Since the primary NPs coexisted in the bulk and on the spherical surfaces together with complete core-shell particles, the formation of those likely occurred at a varying rate in the process. Passing 2 hours of reaction time, the primary NPs were no longer visible (FIG. 1E). The core-shell particles still had diameters of 700 nm-1.5 μm with smooth surfaces that also remained after reaction times reaching 24 hours (FIG. 1F).

Cross-sections of particles collected after 24 hours were prepared by embedding the particles in resin followed by polishing. As can be seen in FIGS. 2A-2B, the hollow cores remained throughout the reaction and the shell thickness of the particles was estimated to 250 nm. Comparing this to the initial shells in FIGS. 1A-2B, where the particle shells were composed of a single layer of primary NPs, indicates that shells were thickened during the reaction by the adsorption of several layers of NPs. Different connections between aggregated core-shell particles were noted when observing the cross-sections. Some particles were only connected by the shells (FIG. 2A), whereas others had cores that were in direct connection with the cores of adjacent particles through their coalescence (FIG. 2B).

To determine the specific role of the ions, the effect of Sr²⁺ and Mg²⁺ was compared separately to investigate how the ionic radius and the concentration were reflected in the characteristics of the primary NPs and the resulting properties of the core-shell particles synthesized at 100° C.

Without substituting ions flake-like structures formed with a morphology resembling that of HA (Ca₁₀(PO₄)₆(OH)₂) (FIG. 4A), which was confirmed in XRD. Using only Sr²⁺ in the synthesis did not result in the formation of core-shell particles (FIG. 4B). Replacing Sr²⁺ with Mg²⁺ showed that Mg²⁺ alone could induce the formation of calcium phosphate core-shell particles. The morphology of the particles was similar to those in FIGS. 1A-1F, but they had slightly smaller diameters ranging between 400-800 nm (FIG. 4C).

The embodiments described above are to be understood as a few illustrative examples of the present invention. It will be understood by those skilled in the art that various modifications, combinations, and changes may be made to the embodiments without departing from the scope of the present invention. In particular, different part solutions in the different embodiments can be combined in other configurations, where technically possible. The scope of the present invention is, however, defined by the appended claims.

What is claimed is:

1. A method of manufacturing hollow calcium phosphate particles having a mean diameter ranging from 400 nm to 1.5 μm, wherein the hollow calcium phosphate particles comprise a respective shell comprising calcium, phosphate, water, magnesium, and strontium, wherein:
a Ca:P atomic ratio is 0.9-1.3;
a Sr²⁺ content is 1-6 wt %;
a Mg²⁺ content is 5-10 wt %; and
a Ca²⁺ content is 15-25 wt %,
wherein the method comprises:
forming a first salt solution comprising 0.5-1.5 mM calcium, 0.2-1.0 mM strontium, and 0.2-1.0 mM magnesium, and a second salt solution comprising 5.0-15 mM phosphate;
mixing the first and the second salt solutions to form a mixed solution at 15-25° C.;
heating the mixed solution to equal to or above 70° C. at a rate of 1-20° C./min forming a heated solution; and
precipitating particles from the heated solution at 70-100° C. for a duration of 1 min to 24 h.

2. The method according to claim 1, further comprising retrieving and washing the particles.

3. The method according to claim 1, wherein heating the mixed solution comprises heating the mixed solution to a temperature selected within a range of from 70 to 100° C.

4. The method according to claim 1, heating the mixed solution comprises heating the mixed solution to a temperature selected within a range of from 70 to 100° C. for a duration of 5 min to 3 h.

5. The method according to claim 1, wherein the first salt solution comprises 0.9 mM calcium, 0.6 mM strontium and 0.5 mM magnesium.

6. The method according to claim 1, wherein the second salt solution comprises 10 mM phosphate.

7. The method according to claim 1, wherein the ratio between Ca:P in the mixed solution is around 0.09.

8. A method of treating or preventing caries and/or dentin hypersensitivity and/or enamel demineralization or decalcification defects in a subject, the method comprises administering hollow calcium phosphate particles to a surface of at least one tooth of the subject, wherein the hollow calcium phosphate particles have a mean diameter ranging from 400 nm to 1.5 μm, and comprise a respective shell comprising calcium, phosphate, water, magnesium, and strontium, and
wherein:
a Ca:P atomic ratio is 0.9-1.3;
a Sr²⁺ content is 1-6 wt %;
a Mg²⁺ content is 5-10 wt %; and
a Ca²⁺ content is 15-25 wt %.

9. The method according to claim 8, wherein the decalcification defects are white spot lesions.

10. A method of treating or preventing caries and/or dentin hypersensitivity and/or enamel demineralization or decalcification defects in a subject, the method comprises administering a composition comprising a paste-forming compound and hollow calcium phosphate particles to a surface of at least one tooth of the subject, wherein the hollow calcium phosphate particles have a mean diameter ranging from 400 nm to 1.5 μm, and comprise a respective shell comprising calcium, phosphate, water, magnesium, and strontium, and
wherein:
a Ca:P atomic ratio is 0.9-1.3;
a Sr²⁺ content is 1-6 wt %;
a Mg²⁺ content is 5-10 wt %; and
a Ca²⁺ content is 15-25 wt %.

11. The method according to claim 10, wherein the decalcification defects are white spot lesions.
